(19) United States
(12) Patent Application Publication
(10) Pub. No.: US 2011/0144724 A1
(43) Pub. Date: Jun. 16, 2011

(54) Phototherapy Apparatus And Method For Tissue Repair

(75) Inventors: Brian Pryor, Newark, DE (US); Sean Xiaolu Wang, Wilmington, DE (US)

(73) Assignee: BWT PROPERTY, INC., Newark, DE (US)

(21) Appl. No.: 12/964,786

(22) Filed: Dec. 10, 2010

Related U.S. Application Data
(60) Provisional application No. 61/285,771, filed on Dec. 11, 2009.

(51) Int. Cl. A61N 5/06

(52) U.S. Cl. 607/88

(57) ABSTRACT

Apparatus and method for applying phototherapy treatment after the use of a bio-scaffold or a bio-implant. Bio-scaffolds are used to regenerate tissue and help the body use its own cell in tissue repair. Phototherapy treatment promotes and speeds up this process by enhancing cell proliferation and differentiation, collagen production/synthesis, fibroblast activity, etc.

### REFERENCE TO RELATED APPLICATION

This application claims an invention which was disclosed in Provisional Patent Application Number 61/285,771, filed Dec. 11, 2009, entitled “PHOTOTHERAPY APPARATUS AND METHOD FOR TISSUE REPAIR”. The benefit under 35 USC § 119(e) of the above mentioned United States Provisional Applications is hereby claimed, and the aforementioned application is hereby incorporated herein by reference.

### FIELD OF THE INVENTION

This invention relates to phototherapy apparatus and method for tissue repair.

### BACKGROUND

Tissue reconstruction has traditionally used synthetic or naturally occurring materials to restore or improve function of organs and tissues afflicted with birth defects or the ravages of injury, disease, and age. Biological scaffolds (bio-scaffolds, bio-implants) have become an integral part of surgical tissue reconstruction in recent years. The scaffolding materials serve as guides for cells to migrate, proliferate and synthesize new extracellular matrices, as well as provide mechanical support during repair of injured tissue (e.g. bone tissue, cardiac tissue, nervous tissue, and ocular tissue). An independent area of endeavor concerns phototherapy, where light energy is used to trigger the natural repair mechanisms carried out by the body to promote and enhance tissue healing. Numerous studies have been conducted which demonstrate the effectiveness of these two techniques on tissue repair. Yet none of them teach or suggest combining these two treatment methods together to achieve a synergetic treatment effect.

### SUMMARY OF THE INVENTION

It is thus the overall goal of the present invention to provide an improved apparatus and method for tissue repair, which applies phototherapy treatment after the use of a bio-scaffold or a bio-implant. The bio-scaffold is used to help the body to use its own cell in tissue repair. The phototherapy treatment promotes and speeds up this process by enhancing cell proliferation and differentiation, collagen production/synthesis, fibroblast activity, etc.

### BRIEF DESCRIPTION OF THE FIGURES

The accompanying FIGURE, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one exemplary embodiment of the phototherapy apparatus and method for tissue repair.

Skilled artisans will appreciate that elements in the FIGURE are illustrated for simplicity and clarity and have not necessarily been drawn to scale. For example, the dimensions of some of the elements in the FIGURE may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to phototherapy apparatus and method for tissue repair. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

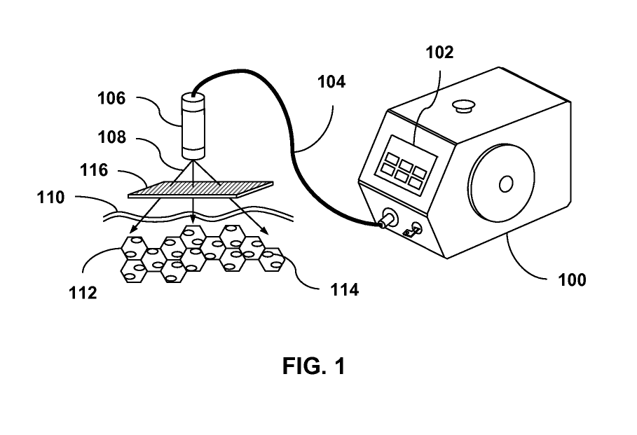

One exemplary embodiment of the present invention is shown in FIG. 1. Phototherapy is applied to the biological tissue 114 where a bio-scaffold 112 is implanted. The scaffold 112 may be made of degradable surgical materials, which serve as guides for cells to migrate, proliferate and synthesize new extracellular matrices, as well as provide mechanical support during repair of injured tissue. It is also possible to use bioactive materials as the scaffold to deliver growth factors/signals, to deliver cells, or to direct the three-dimensional orientation of cells. The scaffold 112 may also be engineered to deliver DNA locally to transduce cells to become bioreactors for production of proteins. The therapeutic light 108 is produced by a diode laser light source 100 and delivered through an optical fiber 104 to an output wand 106. The output wand 106 controls the intensity of the therapeutic light 108, which is applied onto the biological tissue 114. The laser light source 100 may comprise a plurality of diode lasers with different output wavelengths. The output wavelength of the laser light source 100 preferably falls in the near infrared (NIR) region so that the laser light can penetrate through the skin 110 of the patient to treat the inner tissue. A touch-screen based user interface 102 allows the user to control the parameters (e.g. output power, wavelength, treatment time, and pulsing parameters) of the laser light source 100.

The therapeutic light 108 can provide the following beneficial impacts in tissue repair: (i) Phototherapy stimulates white blood cell activity, which plays key roles in clearing out damaged cells; (ii) Phototherapy accelerates macrophage activity in phagocytosis, growth factor secretion and stimulation of collagen synthesis; (iii) The significant angiogenesis that occurs with laser therapy promotes revascularization with subsequent improvement in perfusion and oxygenation; (iv) Light stimulation increases fibroblast numbers and fibroblast-mediated collagen production; (v) Laser-stimulated acceleration of epithelial cell regeneration speeds up wound healing; minimizes scarring, and reduces infection opportunities; (vi) The NIR laser light can increase growth-phase-specific DNA synthesis in normal fibroblasts, muscle cells, osteoblasts and mucosal epithelial cells; (vii) Infrared-induced increases in Nitric Oxide, ATP and other compounds that stimulate higher activity in cell proliferation and differentiation into mature cells; (viii) By increasing the amount of collagen production/synthesis and by increasing the intra and inter-molecular hydrogen bonding in the collagen molecules, laser therapy contributes to improved tensile strength. All these beneficial effects combine to achieve an accelerated healing rate for the biological tissue 114.

By combining the bio-scaffold treatment with the phototherapy treatment, the healing speed of the damaged biological tissue can be greatly improved. In addition, a beam shaping component 116, such as a hologram, a micro-lens array, or a diffraction grating, may be used to modulate the wavefront of the laser beam to produce a non-uniform illumination pattern in the biological tissue. This illumination pattern is aligned with the orientation of the bio-scaffold to guide the migration, proliferation, differentiation, and the three-dimensional orientation of cells to further improve the healing result. For example, the alignment of the newly-formed fibroblasts can be controlled through mechanical guidance of the bio-scaffold and optical guidance of the therapeutic light to produce enhanced tensile strength.

In the foregoing specification, specific embodiments of the present invention have been described. However, one of ordinary skill in the art appreciates that various modifications and changes can be made without departing from the scope of the present invention as set forth in the claims below. The numerical values cited in the specific embodiment are illustrative rather than limiting. Accordingly, the specification and FIGURE are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A method for repairing a damaged biological tissue, the method comprising the steps of:
implanting a biological scaffold at a position of the damaged biological tissue; and
treating the damaged biological tissue with therapeutic light after implanting said biological scaffold.

2. The method of claim 1, wherein said therapeutic light is produced by a diode laser light source.

3. The method of claim 2, wherein said diode laser light source has an output wavelength in the near infrared (NIR) region.

4. The method of claim 2, wherein said diode laser light source comprises a plurality of diode lasers with different output wavelengths.

5. The method of claim 1, further comprising a step of modulating said therapeutic light with a beam shaping component to produce a non-uniform illumination pattern in the biological tissue.

6. The method of claim 5, wherein the beam shaping component comprises at least one of a hologram, a micro-lens array, or a diffraction grating.
